(19) United States
(12) Patent Application Publication
(10) Pub. No.: US 2003/0220287 A1
(43) Pub. Date: Nov. 27, 2003

(54) Antisense nucleic acids

(76) Inventors: M. Ian Phillips, Gainesville, FL (US); Dagmara Mohuczy, Gainesville, FL (US)

Correspondence Address: Stanley A. Kim, Ph.D., Esq., Akerman Senterfitt, Suite 400, 222 Lakeview Avenue, West Palm Beach, FL, 33402-3188, US

(21) Appl. No.: 10/402,099

(22) Filed: Mar. 28, 2003

Related U.S. Application Data
(60) Provisional application No. 60/368,332, filed on Mar. 28, 2002.

(51) Int. Cl. C12Q 1/68; C07H 21/04; A61K 48/00

(52) U.S. Cl. 514/44; 435/6; 435/320.1; 536/23.5

(57) ABSTRACT

Antisense oligonucleotides sequences that inhibit expression of anthrax toxin receptor (ATR) mRNA and human tumor endothelial marker 8 have been designed and constructed. The antisense oligonucleotides may be used to inhibit anthrax infection of host cells as well as for treating cancerous tumors. Introducing such antisense oligonucleotides into a cell decreases ATR expression and decreases tumor cell viability in vitro. Methods for discovering other oligonucleotides with the same activity are taught, as are uses of the antisense molecules for treatment of diseases.

### CROSS REFERENCE TO RELATED APPLICATION

This application claims priority from U.S. provisional patent application Ser. No. 60/368,332, filed Mar. 28, 2002.

### FIELD OF THE INVENTION

The invention relates to the fields of molecular biology, microbiology, oncology, and gene therapy. More particularly, the invention relates to compositions and methods for inhibiting expression of a nucleic acid encoding Bacillus anthracis toxin receptor and/or tumor endothelial marker 8 polypeptide.

### BACKGROUND

Microbial pathogens often exploit host cellular molecules to cause pathology. Among these for example, Bacillus anthracis produces a toxin known to bind cells via the anthrax toxin receptor (ATR). The anthrax toxin includes three different components that are secreted into the bloodstream of an infected animal: protective antigen (PA), edema factor (EF), and lethal factor (LF). PA binds a cell via the ATR, and then creates a pore that allows EF and LF to enter the cytoplasm and cause cellular pathology. More specifically, after binding to ATR, PA is cleaved into two fragments by a furin-like protease. The amino-terminal fragment, PA₂₀, dissociates into the extracellular milieu allowing the carboxy-terminal fragment, PA₆₃ to heptamerize and bind to LF and/or EF, forming the toxin that penetrates and kills the cell. This heptameric complex inserts into the membrane to form a pore allowing translocation of bound EF and LF across the endosomal membrane to the cytosol. Once inside the cell, the catalytic region of EF binds endogenous calmodulin and the binding causes a major conformational change in the catalytic domain. The enzymatic core of EF then catalyzes the conversion of adenosine triphosphate to cyclic adenosine monophosphate causing overproduction of the monophosphate. As a result, cell death and edema occur.

ATR has been shown to be present on cells from several different tissues including the central nervous system, heart, lung, and lymphocytes. It is as a type I transmembrane protein predicted to consist of 368 amino acids. ATR contains a single extracellular von Willebrand factor type A (VWA) domain, located between residues 44 and 216, that binds directly to B. anthracis PA (Bradley K. A. et al., Nature 414: 225-229, 2001). VWA domains are structurally conserved domains important for mediating protein-protein interactions.

Interestingly, ATR was recently indicated to be encoded by the tumor endothelial marker 8 (TEM8) gene (Bradley and Young, Biochemical Pharmacology 65: 309-314, 2003). TEM8 is thought to be involved in angiogenesis. It is expressed at significantly higher levels in human tumor endothelium cells than in normal endothelium (Genbank Accession No. AF279145). A mouse counterpart (mTEM8) has been identified and shown to be abundantly expressed in tumor vessels as well as in the vasculature of the developing mouse embryo (Carson-Walter et al., Cancer Res. 61:6649-6655, 2001). Thus, ATR/TEM8 appear to be a target of clinical significance. For example, the development of techniques for modulating expression of ATR/TEM8 should find use in treating anthrax infection and diseases associated with angiogenesis (e.g., cancer).

### SUMMARY OF THE INVENTION

The invention relates to the development of antisense nucleic acids that may be useful for inhibiting infection of human cells by anthrax bacterium (B. anthracis). Antisense nucleic acids may be used to prevent uptake of anthrax toxin by the cells by inhibiting ATR/ITEM8 expression. Introducing such antisense nucleic acid to cells significantly reduced ATR expression in the cells. Reducing ATR expression prevents binding of the anthrax toxin to host cells and resultant cellular pathology. Additionally, introducing antisense nucleic acids that inhibit ATR/TEM8 expression to cancer cells significantly reduced viability of the cells. Thus, the antisense nucleic acids of the invention might be employed to treat anthrax infection as well as cancer.

Accordingly, the invention features a composition for inhibiting ATR/TEM8 expression in a cell. The composition includes a purified antisense nucleic acid that hybridizes under stringent hybridization conditions to a polynucleotide that encodes a ATR and/or TEM8. Such antisense nucleic acids include, e.g., those listed herein as SEQ ID NOS: 1-17. Examples of cells that express ATR and/or TEM8 include human cells (e.g., a tumor cell).

Also within the invention is a vector including a nucleic acid sequence that encodes an antisense nucleic acid that hybridizes under stringent hybridization conditions to a polynucleotide that encodes ATR and/or TEM8.

Another aspect of the invention features a method of modulating ATR or TEM8 expression in a cell. The method includes the steps of providing a cell expressing a molecule selected from ATR and TEM8; and contacting the cell with an agent that modulates expression of the molecule in the cell. In preferred variations of the method, the agent causes expression in the cell of an antisense nucleic acid that hybridizes under stringent hybridization conditions to a polynucleotide that encodes the molecule.

The invention further features a method of modulating tumor cell viability. This method includes the steps of providing a tumor cell expressing TEM8 and administering to the tumor cell a composition comprising an agent that modulates expression of TEM8 in the cell. In one variation of this method, the agent causes expression in the cell of an antisense nucleic acid that hybridizes under stringent hybridization conditions to a polynucleotide that encodes TEM8.

Unless otherwise defined, all technical terms used herein have the same meaning as commonly understood by one of ordinary skill in the art to which this invention belongs.

Use of the term “expression” refers to transcription and/or translation of a nucleic acid molecule to produce a complementary nucleic acid or a polypeptide.

As used herein, a “nucleic acid” or a “nucleic acid molecule” means a chain of two or more nucleotides such as RNA (ribonucleic acid) and DNA (deoxyribonucleic acid). A “purified” nucleic acid molecule is one that has been substantially separated or isolated away from other nucleic acid sequences in a cell or organism in which the nucleic acid naturally occurs (e.g., 30, 40, 50, 60, 70, 80, 90, 95, 96, 97, 98, 99, 100% free of contaminants). The term includes, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced by polymerase chain reaction (PCR), nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

As used herein, “protein” or “polypeptide” are used synonymously to mean any peptide-linked chain of amino acids, regardless of length or post-translational modification, e.g., glycosylation or phosphorylation.

When referring to hybridization of one nucleic to another, “low stringency conditions” means in 10% formamide, 5× Denhart's solution, 6× SSPE, 0.2% SDS at 42° C., followed by-washing in 1× SSPE, 0.2% SDS, at 50° C.; “moderate stringency conditions” means in 50% formamide, 5× Denhart's solution, 5× SSPE, 0.2% SDS at 42° C., followed by washing in 0.2× SSPE, 0.2% SDS, at 65° C.; and “high stringency conditions” means in 50% formamide, 5× Denhart's solution, 5× SSPE, 0.2% SDS at 42° C., followed by washing in 0.1× SSPE, and 0.1% SDS at 65° C. The phrase “stringent hybridization conditions” means low, moderate, or high stringency conditions.

As used herein, “sequence identity” means the percentage of identical subunits at corresponding positions in two sequences when the two sequences are aligned to maximize subunit matching, i.e., taking into account gaps and insertions. When a subunit position in both of the two sequences is occupied by the same monomeric subunit, e.g., if a given position is occupied by an adenine in each of two DNA molecules, then the molecules are identical at that position. For example, if 7 positions in a sequence 10 nucleotides in length are identical to the corresponding positions in a second 10-nucleotide sequence, then the two sequences have 70% sequence identity. Sequence identity is typically measured using sequence analysis software (e.g., Sequence Analysis Software Package of the Genetics Computer Group, University of Wisconsin Biotechnology Center, 1710 University Avenue, Madison, Wis. 53705).

As used herein, the term “vector” refers to a nucleic acid molecule capable of transporting another nucleic acid to which it has been linked. One type of vector is an episome, i.e., a nucleic acid capable of extra-chromosomal replication. Another type of vector is one that integrates into the host genome. Preferred vectors are those capable of autonomous replication and/expression of nucleic acids to which they are linked. Vectors capable of directing the expression of genes to which they are operatively linked are referred to herein as “expression vectors.”

A first nucleic acid sequence is “operably” linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein coding regions, in reading frame.

A cell, tissue, or organism into which has been introduced a foreign nucleic acid, such as a recombinant vector, is considered “transformed,” “transfected,” or “transgenic.” A “transgenic” or “transformed” cell or organism (e.g., a mammalian cell) also includes progeny of the cell or organism. For example, a mammal transgenic for antisense nucleic acid that hybridizes to an mRNA encoding ATR and/or TEM8 polypeptide is one in which antisense nucleic acid has been introduced.

Although methods and materials similar or equivalent to those described herein can be used in the practice or testing of the present invention, suitable methods and materials are described below. All publications, patent applications, patents and other references mentioned herein are incorporated by reference in their entirety. The particular embodiments discussed below are illustrative only and not intended to be limiting.

### BRIEF DESCRIPTION OF THE DRAWINGS

The above and further advantages of this invention may be better understood by referring to the following description taken in conjunction with the accompanying drawings, in which:

FIG. 1 is a graph showing a decrease in ATR/TEM8 mRNA in human lung fibroblast CCD-Lu39 cells 24 hours after transfection with antisense oligonucleotides to ATR/TEM8 mRNA.

FIG. 2 is a graph showing a decrease in ATR/TEM8 mRNA in human lung fibroblast CCD-Lu39 cells 48 hours after transfection with antisense oligonucleotides to ATR/TEM8 mRNA.

### DETAILED DESCRIPTION

The invention provides compositions and methods for preventing uptake of anthrax toxin by host cells by inhibiting expression of a nucleic acid that encodes ATR and/or TEM8 polypeptide in a cell. The invention also provide compositions and methods for inhibiting tumor cell viability by inhibiting expression of a nucleic acid that encodes ATR and/or TEM8 polypeptide in a cell. Purified nucleic acids (e.g., antisense oligonucleotides) that hybridize to a nucleic acid (e.g., mRNA) encoding these polypeptides are useful for preventing uptake of anthrax toxin into cells as well as inhibiting tumor cell viability.

The below described preferred embodiments illustrate adaptations of these compositions and methods. Nonetheless, from the description of these embodiments, other aspects of the invention can be made and/or practiced based on the description provided below.

Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

Antisense Targets

The invention relates to methods and compositions for inhibiting expression of nucleic acids involved in a B. anthracis infection, including those which encode ATR. As binding of PA to ATR is required for infection of a cell by B. anthracis, blocking expression of ATR will block infection of the cell by the bacterium. An important aspect of the invention, therefore, relates to the inhibition of expression of ATR using antisense nucleic acids (e.g., SEQ ID NOS: 1-17) that hybridize to nucleic acids (e.g., mRNA) encoding ATR protein. The invention also relates to methods and compositions for inhibiting TEM8 expression. Inhibition of TEM8 expression in cancer cell using an antisense strategy reduces the viability of cancer cells.

Nucleic Acids Encoding ATR and TEM8

The invention provides compositions for inhibiting expression of a nucleic acid (e.g., mRNA) that encodes ATR and/or TEM8 polypeptide in a cell (e.g., a human cell such as a human cancer cell). Such compositions include a purified antisense nucleic acid (e.g., DNA oligonucleotide) that hybridizes under stringent hybridization (e.g., high stringency) conditions to a nucleic acid that encodes ATR and/or TEM8 polypeptide. Expression of a variety of different mRNA sequences that encode ATR and/or TEM8 polypeptides may be inhibited using compositions and methods of the invention. For example, mRNA sequences encoding ATR and/or TEM8 polypeptide include the mRNA sequences of Genbank Accession Nos. NM_{—}018153, NM_{—}053034, AF421380, AF279145, NM_{—}032208, and NT_{—}022354.

Compositions for Inhibiting ATR/TEM8 Expression

The invention provides purified antisense nucleic acids (e.g., DNA oligonucleotides) that hybridize under stringent hybridization conditions to a nucleic acid (e.g., mRNA) that encodes ATR and/or TEM8 polypeptide. The purified antisense nucleic acids are useful for inhibiting expression of ATR and TEM8 polypeptides. Antisense nucleic acid molecules within the invention are those that specifically hybridize under cellular conditions to cellular mRNA and/or genomic DNA encoding an ATR and/or TEM8 protein in a manner that inhibits expression of the ATR and/or TEM8 protein, e.g., by inhibiting transcription and/or translation. The binding may be by conventional base pair complementarity, or, for example, in the case of binding to DNA duplexes, through specific interactions in the major groove of the double helix.

An antisense nucleic acid according to the invention can be any nucleic acid that hybridizes under stringent hybridization conditions to a DNA or mRNA molecule encoding ATR and/or TEM8 polypeptide. In illustrative embodiments, antisense oligonucleotides may be prepared which are complementary nucleic acid sequences that can recognize and bind to target genes or the transcribed mRNA, resulting in the arrest and/or inhibition of DNA transcription or translation of the mRNA. These oligonucleotides can be expressed within a host cell that normally expresses a specific mRNA encoding an ATR and/or TEM8 polypeptide to reduce or inhibit the expression of this mRNA. Thus, the oligonucleotides may be useful for reducing the level of polypeptide in a cell.

In preferred embodiments, an antisense oligonucleotide contains a sequence of at least seven, at least eight, at least nine, at least ten, at least eleven, at least twelve, at least thirteen or at least fourteen contiguous bases from SEQ ID NO:1, SEQ ID NO:2, SEQ ID NO:3, SEQ ID NO:4, SEQ ID NO:5, SEQ ID NO6, :SEQ ID NO:7, SEQ ID NO8, :SEQ ID NO:9, SEQ ID NO:10, SEQ ID NO: 11, SEQ ID NO:12, SEQ ID NO:13, SEQ ID NO:14, SEQ ID NO:15, SEQ ID NO:16, or SEQ ID NO:17. A more preferred antisense nucleic acid for inhibiting expression of a nucleic acid that encodes ATR and/or TEM8 polypeptide is the nucleic acid sequence of SEQ ID NO:7. Cells transfected with this antisense oligonucleotide demonstrate a decrease in mRNA encoding ATR and/or TEM8. Furthermore, tumor cells transfected with this antisense oligonucleotide show a decrease in viability.

Antisense approaches involve the design of oligonucleotides (either DNA or RNA) that are complementary to mRNA encoding ATR and/or TEM8. General approaches to constructing oligomers useful in antisense therapy have been reviewed, for example, by Van der Krol et al. Biotechniques 6:958-976, 1988; and Stein et al. Cancer Res 48:2659-2668, 1988. The antisense oligonucleotides may inhibit expression of ATR and/or TEM8 polypeptide, for example, by binding to Atr/Tem8 mRNA transcripts and preventing translation. Absolute complementarity, although preferred, is not required. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex. Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. (Wagner, R. Nature 372:333, 1994). Therefore, oligonucleotides complementary to either the 5′ or 3′ regions of a nucleic acid encoding ATR and/or TEM8 could be used in an antisense approach to inhibit translation of endogenous ATR and/or TEM8 mRNA. With respect to antisense DNA, oligodeoxyribonucleotides that hybridize to a region of an ATR and/or TEM8-encoding nucleotide sequence containing an AUG start codon, are preferred. Whether designed to hybridize to the 5′, 3′ or coding region of mRNA encoding ATR and/or TEM8, antisense nucleic acids should be at least six nucleotides in length, and are preferably less than about 100 and more preferably less than about 50, 25, or 17 nucleotides in length.

Oligonucleotides in their natural form as phosphodiesters are subject to rapid degradation in the blood, intracellular fluid or cerebrospinal fluid by exo- and endonucleases. Exemplary nucleic acid molecules for use as antisense oligonucleotides are phosphoramidate, phosphothioate and methylphosphonate analogs of DNA (see, e.g., U.S. Pat. Nos. 5,176,996; 5,264,564; and 5,256,775). The most widely used modified antisense oligonucleotides are phosphorothioates, where one of the oxygen atoms in the phosphodiester bond between nucleotides is replaced with a sulfur atom. These phosphorothioate antisense oligonucleotides have greater stability in biological fluids than normal oligos and are preferred antisense nucleic acids within the invention.

Regardless of the choice of target sequence, it is preferred that in vitro studies are first performed to quantify the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

Antisense oligonucleotides of the invention may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxyethyl) uracil, 5-carboxymethylaminomethyl-2-thiouridine, 5-carboxymethylaminomethyluracil, dihydrouricil, beta-D-galactosylqueosine, inosine, N6-isopentenyladenine, 1-methylguanine, 1-methylinosine, 2,2-idimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Antisense oligonucleotides of the invention may also comprise at least one modified sugar moiety selected from the group including but not limited to arabinose, 2-fluoroarabinose, xylulose, and hexose; and may additionally include at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-6641, 1987). Such an oligonucleotide can be a 2′-0-methylribonucleotide (Inoue et al., Nucl. Acids Res. 15:6131-6148, 1987), or a chimeric RNA-DNA analogue (Inoue et al., FEBS Lett. 215:327-330, 1987).

Ribozyme molecules designed to catalytically cleave Atr and/or Tem8 mRNA transcripts can also be used to prevent translation of Atr and/or Tem8 mRNA and expression of ATR and/or TEM8 polypeptides (See, e.g., PCT Publication No. WO 90/11364, published Oct. 4, 1990; Sarver et al., Science 247:1222-1225, 1990; and U.S. Pat. No. 5,093,246). While ribozymes that cleave mRNA at site specific recognition sequences can be used to destroy Atr and/or Tem8 mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach (1988) Nature 334:585-591. Preferably the ribozyme is engineered so that the cleavage recognition site is located near the 5′ end of Atr and/or Tem8 mRNA; i.e., to increase efficiency and minimize the intracellular accumulation of non-functional mRNA transcripts. Ribozymes within the invention can be delivered to a cell using a vector as described below.

Alternatively, endogenous Atr and/or Tem8 gene expression might be reduced by targeting deoxyribonucleotide sequences complementary to the regulatory region of the Atr and/or Tem8 gene (i.e., the Atr and/or Tem8 promoter and/or enhancers) to form triple helical structures that prevent transcription of the Atr and/or Tem8 gene in target cells. (See generally, Helene, C. Anticancer Drug Des. 6(6):569-84, 1991; Helene, C., et al. Ann. N.Y. Acad. Sci. 660:27-36, 1992; and Maher, L. J. Bioassays 14(12):807-15, 1992).

Nucleic acid molecules to be used in triple helix formation for the inhibition of transcription are preferably single-stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Another technique that may be employed to modulate ATR and/or TEM8 expression is RNA interference (RNAi, Chuang and Meyerowicz, Proc. Nat'l Acad. Sci. USA, 97:4985, 2000). RNAi induces gene-specific suppression through sequence-specific degradation of homologous gene transcripts (P. Sharp, Genes & Development 13:139-141, 1999; Bernstein et al., RNA 7:1509-1521, 2001; and Hutvagner and Zamore, Curr. Opin. Genet. Dev. 12:225-232, 2002). In this technique, double-stranded RNA (dsRNA)-expressing constructs are introduced into a cell and the dsRNA molecules are metabolized to 21-23 nucleotide small interfering RNAs (siRNA). By selecting appropriate sequences (e.g. , those corresponding to Atr and/or Tem8), expression of dsRNA can interfere with accumulation of (e.g., degradation of) endogenous mRNA encoding a target protein (e.g., ATR and/or TEM8). Efficient introduction of siRNAs into cells in vitro may be performed using a number of technologies, including lipid-based transfection techniques as well as Nucleofector™ technology (Amaxa, Cologne, Germany). Gene silencing mediated by siRNAs in mammalian cells is described in Scherr et al., Curr. Med. Chem. 10:245-256, 2003; and Doi et al., Curr. Biol. 13:41-46, 2003.

Additional methods of gene silencing include the use of messenger RNA-antisense DNA interference (D-RNAi) and peptide nucleic acid (PNA) oligonucleotide technologies. D-RNAi is a posttranscriptional mechanism of silencing gene expression by the introduction of mRNA-DNA hybrids to a cell. D-RNAi has been shown to effect long-term gene silencing and is discussed in Lin SL Curr. Cancer Drug Targets 1:241-247, 2001; and Chen et al., Exp. Biol. Med. 227:75-87, 2002. PNA oligonucleotides hybridize to complementary DNA or RNA and inhibit transcription and translation of target genes by this hybridization. PNA oligos have been successfully used as an antisense agent in cultured cells as well as in vivo (Pooga and Langel Curr. Cancer Drug Targets 1:231-239, 2001).

Antisense RNA and DNA, ribozyme, and triple helix molecules of the invention may be prepared by any method known in the art for the synthesis of DNA and RNA molecules. These include techniques for chemically synthesizing oligodeoxyribonucleotides (e.g., by use of an automated DNA synthesizer such as are commercially available from Biosearch, Applied Biosystems, etc.) and oligoribonucleotides well known in the art such as for example solid phase phosphoramide chemical synthesis. As examples, phosphorothioate oligonucleotides may be synthesized by the method of Stein et al. (Nucl. Acids Res. 16:3209, 1988), and methylphosphonate oligonucleotides can be prepared by use of controlled pore glass polymer supports (Sarin et al., Proc. Natl. Acad. Sci. U.S.A. 85:7448-7451, 1988), etc.

Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Antisense constructs can be delivered, for example, as an expression plasmid which, when transcribed in the cell, produces RNA which is complementary to at least a unique portion of the cellular mRNA which encodes an ATR and/or TEM8 protein. Alternatively, the antisense construct can take the form of an oligonucleotide probe generated in vitro or ex vivo which, when introduced into an ATR/TEM8-expressing cell, causes inhibition of ATR and/or TEM8 expression by hybridizing with an mRNA and/or genomic sequences coding for ATR and/or TEM8. Such oligonucleotide probes are preferably modified oligonucleotides that are resistant to endogenous nucleases, e.g. exonucleases and/or endonucleases, and are therefore stable in vivo.

Cells Containing Nucleic Acids Encoding ATR and TEM8

The invention provides compositions and methods for inhibiting expression of a nucleic acid that encodes ATR and/or TEM8 polypeptide in a cell. Compositions of the invention may be introduced into any cell that contains a nucleic acid encoding ATR and TEM8. Such cells include animal cells, preferably human cells. Human cells containing nucleic acids encoding ATR and/or TEM8 include those cells cultured in vitro as well as those within a human being. In some applications, compositions of the invention are introduced into tumor cells (e.g., human tumor cells). Such tumor cells include those cultured in vitro as well as those within a human tumor. An example of a human tumor is a tumor located within a human being. Antisense nucleic acids of the invention may be used to treat tumors by introducing the nucleic acid into one or more tumor cells and effecting a decrease in tumor cell viability, thereby killing the tumor. Examples of tumors that may be treated using compositions and methods of the invention include cervical cancers and adenocarcinomas, as well as any others that express TEM8.

Modulating ATR/TEM8 Levels In A Cell

Within the invention is a method for modulating ATR and/or TEM8 levels in a cell. Methods of modulating ATR and/or TEM8 levels in a cell can be used to enhance or inhibit expression of ATR and/or TEM8 in a cell. One example of a method of modulating ATR and/or TEM8 levels in a cell includes the steps of providing a cell and administering to the cell a composition including an agent that inhibits expression of ATR and/or TEM8 in the cell. The agent can be a purified antisense nucleic acid that hybridizes under stringent hybridization conditions to a nucleic acid that encodes ATR and/or TEM8. A number of suitable antisense nucleic acids are described above. A purified antisense nucleic acid can be administered to any human cell, including a cell within a human. Techniques and mechanisms of antisense inhibition of gene expression are described in Sazani et al., Curr. Opin. Biotechnol. 13:468-472, 2002; Jansen and Zangemeister-Wittke, Lancet Oncol. 3:672-683, 2002; and Agrawal and Kandimalla Curr. Cancer Drug Targets 1:197-209, 2001.

Purified antisense oligonucleotides of the invention may be administered to a cell (e.g., within a human subject) using any suitable method, including parenteral injection of antisense DNA oligonucleotides to an animal. Additionally, a number of gene therapy technologies may be used to deliver antisense oligonucleotides to cells of an animal. Methods and compositions involving gene therapy vectors are described herein. Such techniques are generally known in the art and are described in methodology references such as Viral Vectors, eds. Yakov Gluzman and Stephen H. Hughes, Cold Spring Harbor Laboratory Press, Cold Spring Harbor, N.Y. , 1988; Retroviruses, Cold Spring Harbor Laboratory Press, Plainview, N.Y. , 2000; Gene Therapy Protocols (Methods in Molecular Medicine), ed. Jeffrey R. Morgan, Humana Press, Totawa, N.J. , 2001; and Molecular Cloning: A Laboratory Manual, 3nd ed., vol. 1-3, ed. Sambrook et al., Cold Spring Harbor Laboratory Press, Cold Spring Harbor, N.Y. , 2001. For a review of liver-directed gene transfer vectors, see Ferry and Heard, Human Gene Ther. 9:1975-1981, 1998.

Because it is often difficult to achieve intracellular concentrations of the antisense sufficient to suppress translation on endogenous mRNAs, a preferred approach utilizes a recombinant DNA construct in which the antisense oligonucleotide is placed under the control of a strong promoter, including a viral promoter or a non-viral promoter. Examples of strong viral and non-viral promoters include cytomegalovirus (CMV), rous sarcoma virus (RSV), simian virus 40 (SV40), human elongation factor 1 α(hEF 1 α), and a hybrid CMV/chicken β actin (CBA) promoter. To achieve high levels of expression, a CBA promoter may be coupled to a woodchuck hepatitis virus post-transcriptional regulatory sequence (WPRE). The use of such a construct to transform mammalian cells will result in the transcription of sufficient amounts of single stranded RNAs that will, for example, form complementary base pairs with the endogenous Atr and/or Tem8 transcripts and thereby prevent translation of mRNA encoding ATR and/or TEM8.

Various techniques using viral vectors for the administration of antisense nucleic acids (e.g., antisense oligonucleotides) to cells are provided for according to the invention. Viruses are naturally evolved vehicles which efficiently deliver their genes into host cells and therefore are desirable vector systems for the delivery of therapeutic genes. Preferred viral vectors exhibit low toxicity to the host cell and produce therapeutic quantities of antisense oligonucleotides. In some applications, preferred viral vectors produce therapeutic quantities of antisense oligonucleotides in a tissue-specific manner (e.g., tumor cells). Viral vector methods and protocols are reviewed in Kay et al. Nature Medicine 7:33-40, 2001; Tal, J., J. Biomed. Sci. 7:279-291, 2000; and Monahan and Samulski, Gene Therapy 7:24-30, 2000.

The rAAV vectors and rAAV virions used in the invention may be derived from any of several AAV serotypes including 1, 2, 3, 4, 5, 6, and 7. Particular AAV vectors and AAV proteins of different serotypes are discussed in Chao et al., Mol. Ther. 2:619-623, 2000; Davidson et al., PNAS 97:3428-3432, 2000; and Xiao et al., J. Virol. 72:2224-2232, 1998. The invention also relates to the use of rAAV virions that have mutations within the virion capsid. For example, suitable rAAV mutants may have ligand insertion mutations for the facilitation of targeting rAAV virions to specific cell types (e.g., tumor cells). The construction and characterization of rAAV capsid mutants including insertion mutants, alanine screening mutants, and epitope tag mutants is described in Wu et al., J. Virol. 74:8635-45, 2000. Pseudotyped rAAV virions that have mutations within the capsid may also be used in compositions and methods of the invention. Pseudotyped rAAV virions contain an rAAV vector derived from a particular serotype that is encapsidated within a capsid containing proteins of another serotype. Techniques involving nucleic acids and viruses of different AAV serotypes are known in the art and are described in Halbert et al., J. Virol. 74:1524-1532, 2000; and Auricchio et al., Hum. Molec. Genet. 10:3075-3081, 2001. Other rAAV virions that can be used in methods of the invention include those capsid hybrids that are generated by molecular breeding of viruses as well as by exon shuffling. See Soong et al., Nat. Genet. 25:436-439, 2000; and Kolman and Stemmer Nat. Biotechnol. 19:423-428, 2001.

Another example of a viral vector that may be used for DNA transfer is adenovirus. Methods for use of recombinant adenoviruses as gene therapy vectors are discussed, for example, in W. C. Russell, Journal of General Virology 81:2573-2604, 2000, and Bramson et al., Curr. Opin. Biotechnol. 6:590-595, 1995. Adenovirus vectors have been shown to be capable of highly efficient gene expression in target cells and allow for a large coding capacity of heterologous DNA. Heterologous DNA in this context may be defined as any nucleotide sequence or gene which is not native to the adenovirus. A preferred form of recombinant adenovirus is a “gutless”, “high-capacity”, or “helper-dependent” adenovirus vector which has all viral coding sequences deleted, and contains the viral inverted terminal repeats (ITRs), therapeutic gene (e.g., an antisense oligonucleotide) sequences (up to 28-32 kb) and the viral DNA packaging sequence. Variants of such recombinant adenovirus vectors such as vectors containing tissue-specific (e.g., tumor-specific) enhancers and promoters operably linked to an antisense oligonucleotide are also within the invention. More than one promoter can be present in a vector. Accordingly, more than one heterologous antisense oligonucleotide can be expressed by a vector.

Additionally, herpes simplex virus (HSV)-based vectors may be used. Methods for use of HSV vectors are discussed, for example, in Cotter and Robertson, Curr. Opin. Mol. Ther. 1:633-644, 1999. HSV vectors deleted of one or more immediate early genes (IE) are non-cytotoxic, persist in a state similar to latency in the host cell, and afford efficient host cell transduction. Recombinant HSV vectors allow for approximately 30 kb of coding capacity. A preferred HSV vector is engineered from HSV type I, and is deleted of the immediate early genes (IE). In some applications (e.g., anti-cancer applications), a preferred HSV vector also contains a tissue-specific (e.g., tumor-specific) promoter operably linked to a antisense oligonucleotide. HSV amplicon vectors may also be used according to the invention. Typically, HSV amplicon vectors are approximately 15 kb in length, possess a viral origin of replication and packaging sequences. More than one promoter can be present in a vector. Accordingly, more than one antisense oligonucleotide can be expressed by a vector.

Viral vectors of the present invention may also include replication-defective lentiviral vectors, including HIV. Methods for use of lentiviral vectors are discussed, for example, in Vigna and Naldini, J. Gene Med. 5:308-316, 2000 and Miyoshi et al., J. Virol. 72:8150-8157, 1998. Lentiviral vectors are capable of infecting both dividing and non-dividing cells and efficient transduction of epithelial tissues of humans. Lentiviral vectors according to the invention may be derived from human and non-human (including SIV) lentiviruses. In certain applications (e.g., anti-cancer applications), preferred lentiviral vector of the present invention may include nucleic acid sequences required for vector propagation in addition to a tissue-specific promoter (e.g., tumor-specific) operably linked to a antisense oligonucleotide. These sequences may include the viral LTRs, primer binding site, polypurine tract, att sites and encapsidation site. The lentiviral vector may be packaged into any suitable lentiviral capsid. The substitution of one particle protein by one from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles. More than one promoter can be present in a vector. Accordingly, more than one antisense oligonucleotide can be expressed by a vector.

The invention also provides for use of retroviral vectors, including MLV-based vectors. Methods for use of retrovirus-based vectors are discussed, for example, in Hu and Pathak, Pharmacol. Rev. 52:493-511, 2000 and Fong et al., Crit. Rev. Ther. Drug Carrier Syst. 17:1-60, 2000. Retroviral vectors according to the invention may contain up to 8 kb of heterologous (therapeutic) DNA, in place of the viral genes. Heterologous may be defined in this context as any nucleotide sequence or gene which is not native to the retrovirus (e.g., antisense oligonucleotides). The heterologous DNA may also include a tissue-specific promoter, an antisense oligonucleotide, and sequences encoding a ligand to a tumor cell-specific receptor. The retroviral particle may be pseudotyped, and may contain a viral envelope glycoprotein from another virus, in place of the native retroviral glycoprotein. The retroviral vector of the present invention may integrate into the genome of the host cell. More than one promoter can be present in a vector. Accordingly, more than one antisense oligonucleotide can be expressed by a vector.

Other viral vectors within the invention are alphaviruses, including Semliki forest virus (SFV) and Sindbis virus (SIN). Methods for use of alphaviruses are described, for example, in Lundstrom, K., Intervirology 43:247-257, 2000 and Perri et al., Journal of Virology 74:9802-9807, 2000. Alphavirus vectors typically are constructed in a format known as a replicon. Such replicons may contain alphavirus genetic elements required for RNA replication, as well as antisense oligonucleotide expression. Heterologous may be defined in this context as any nucleotide sequence or gene which is not native to the alphavirus. Within the alphivirus replicon, the antisense oligonucleotide may be operably linked to a tissue-specific (e.g., tumor-specific) promoter or enhancer. Recombinant, replication-defective alphavirus vectors are capable of high-level heterologous (therapeutic) gene expression, and can infect a wide host cell range. Alphavirus replicons according to the invention may be targeted to specific cell types (e.g., tumor cells) by displaying on their virion surface a functional heterologous ligand or binding domain that would allow selective binding to target cells expressing the cognate binding partner. Alphavirus replicons according to the invention may establish latency, and therefore long-term antisense oligonucleotide expression in the host cell. The replicons may also exhibit transient antisense oligonucleotide expression in the host cell. A preferred alphavirus vector or replicon of the invention is noncytopathic. More than one promoter can be present in a vector. Accordingly, more than one heterologous gene (e.g., antisense oligonucleotide) can be expressed by a vector.

To combine advantageous properties of two viral vector systems, hybrid viral vectors may be used to deliver an antisense oligonucleotide to a subject. Standard techniques for the construction of hybrid vectors are well-known to those skilled in the art. Such techniques can be found, for example, in Sambrook, et al., supra or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retroviral/Adenovirus hybrid vectors are discussed in Zheng et al., Nature Biotechnol. 18:176-186, 2000. Retroviral genomes contained within an Adenovirus may integrate within the host cell genome and effect stable antisense oligonucleotide expression. More than one promoter can be present in a vector. Accordingly, more than one heterologous gene (e.g., antisense oligonucleotide) can be expressed by a vector.

In accordance with the present invention, other nucleotide sequence elements which facilitate expression of the antisense oligonucleotide and cloning of the vector are further contemplated. The presence of enhancers upstream of the promoter or terminators downstream of the coding region, for example, can facilitate expression. In the vectors of the present invention, the presence of elements which enhance tumor cell-specific expression of antisense oligonucleotides may be useful for gene therapy in treating cancerous tumors.

Several non-viral methods for introducing an antisense oligonucleotide into host cells are also within the scope of the invention. For a review of non-viral methods, see Nishikawa and Huang, Human Gene Ther. 12:861-870, 2001. Various techniques employing plasmid DNA for the introduction of an antisense oligonucleotide into cells are provided for according to the invention. Such techniques are generally known in the art and are described in references such as Ilan, Y., Curr. Opin. Mol. Ther. 1:116-120, 1999, Wolff, J. A. , Neuromuscular Disord. 7:314-318, 1997 and Arztl, Z., Fortbild Qualitatssich 92:681-683, 1998. Alternatively, modified antisense molecules, designed to target the desired cells (e.g., antisense linked to peptides or antibodies that specifically bind receptors or antigens expressed on the target cell surface) can be used.

Methods involving physical techniques for introducing an antisense oligonucleotide into a host cell can be adapted for use in the present invention. The particle bombardment method of gene transfer involves a gene gun (e.g., Accell device by Geniva, Madison, Wis.; and Helios gene gun by Biorad, Hercules, Calif.) to accelerate DNA-coated microscopic gold particles into target tissue. Particle bombardment methods are described in Yang et al., Mol. Med. Today 2:476-481 1996 and Davidson et al., Rev. Wound Repair Regen. 6:452-459, 2000. Cell electropermeabilization (also termed cell electroporation) may be employed for antisense oligonucleotide delivery into cells of tissues. This technique is discussed in Preat, V., Ann. Pharm. Fr. 59:239-244 2001 and involves the application of pulsed electric fields to cells to enhance cell permeability, resulting in exogenous polynucleotide transit across the cytoplasmic membrane.

Synthetic gene transfer molecules according to the invention can be designed to form multimolecular aggregates with DNA (harboring antisense oligonucleotide sequence operably linked to a promoter) and to bind the resulting particles to the target cell (e.g., tumor cells) surface in such a way as to trigger endocytosis and endosomal membrane disruption. For example, polymeric DNA-binding cations (including polylysine, protamine, and cationized albumin) can be linked to tumor-specific targeting ligands and trigger receptor-mediated endocytosis into tumor cells. Methods involving polymeric DNA-binding cations are reviewed in Guy et al., Mol. Biotechnol. 3:237-248, 1995 and Garnett, M. C. , Crit. Rev. Ther. Drug Carrier Syst. 16:147-207, 1999. Cationic amphiphiles, including lipopolyamines and cationic lipids, may provide receptor-independent antisense oligonucleotide transfer into target cells (e.g., tumor cells). Liposomes are self-assembling particles of bilipid layers that have been used for encapsulating antisense oligonucleotides for delivery in blood and cell culture. Preformed cationic liposomes or cationic lipids may be mixed with DNA (e.g., oligonucleotides) to generate cell transfecting complexes (e.g., Lipofectamine, Oligofectamine, Invitrogen, Carlsbad, Calif.). Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. Suitable methods can also include use of cationic liposomes as agents for introducing DNA (e.g., antisense oligonucleotide) into cells. For therapeutic gene delivery, DNA may also be coupled to an amphipathic cationic peptide (Fominaya et al., J. Gene Med. 2:455-464, 2000).

Methods that involve both viral and non-viral based components may be used according to the invention. An Epstein Barr virus (EBV) based plasmid for therapeutic gene delivery is described in Cui et al., Gene Therapy 8:1508-1513, 2001. A method involving a DNA/ligand/polycationic adjunct coupled to an adenovirus is described in Curiel, D. T. , Nat. Immun. 13:141-164, 1994. More than one promoter can be present in a vector. Accordingly, more than one antisense oligonucleotide can be expressed by a vector.

Other techniques according to the invention may be based on the use of tumor-specific ligands. Synthetic peptides or polypeptides may be used as ligands in targeted delivery of DNA to tumor-specific receptors. Complexes of protein and ligand or plasmid DNA and ligand mediate DNA transfer into tumor cells.

Methods involving ultrasound contrast agent delivery vehicles may be used in the invention. Such methods are discussed in Newman et al., Echocardiography 18:339-347, 2001 and Lewin et al. Invest. Radiol. 36:9-14, 2001. Gene-bearing microbubbles, when exposed to ultrasound, cavitate and locally release a therapeutic agent. Attachment of a tumor cell-targeting moiety to the contrast agent vehicle may result in site-specific (e.g., tumor) antisense oligonucleotide delivery.

Methods which are well known to those skilled in the art can be used to construct a natural or synthetic matrix that provides support for the delivered agent (antisense oligonucleotide) prior to delivery. See, for example, the techniques described in Murphy and Mooney, J. Period Res., 34:413-9, 1999 and Vercruysse and Prestwich, Crit. Rev. Ther. Drug Carrier Syst., 15:513-55, 1998. The particular type of matrix used can be any suitable matrix for use in the invention. For implantation into an animal subject, preferred matrix will have all the features commonly associated with being “biocompatible”, in that they do not produce an adverse, or allergic reaction when administered to the recipient host. Matrices suitable for use in the invention may be formed from both natural or synthetic materials and may be designed to allow for sustained release of the therapeutic agent over prolonged periods of time. Preferred matrices are those that are biodegradable as these are capable of being reabsorbed.

Delivery of an antisense oligonucleotide, according to the invention, may involve methods of DNA microencapsulation. Microparticles, also known as microcapsules and microspheres, may be used as gene delivery vehicles. They may be delivered in operable form noninvasively to epithelial surfaces for gene therapy. The genes within the microparticles can pass across epithelial barriers and travel to remote sites, via systemic circulation. Microencapsulated gene delivery vehicles may be constructed from low viscosity polymer solutions that are forced to phase invert into fragmented spherical polymer particles when added to appropriate nonsolvents. Methods involving microparticles are discussed in Hsu et al., J. Drug Target 7:313-323, 1999 and Capan et al., Pharm. Res. 16:509-513, 1999.

Administration Of Compositions

The compositions of the invention may be administered to animals (e.g., humans) in any suitable formulation by any conventional technique. Purified antisense nucleic acids may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the composition.

Among delivery routes, parenteral delivery, e.g., by intravenous injection, is sometimes preferred. The compositions may also be administered directly to a target site by, for example, surgical delivery to an internal or external target site, or by catheter to a site accessible by a blood vessel. While several methods of delivery may be employed, nasal sprays may be particularly advantageous for use in treating anthrax infections, as port of entry is frequently through the lungs. Additionally, bronchoalveolar instillation (Koren et al., Am. Rev. Respir. Dis. 139:407-415, 1989) may also be used to deliver compositions of the invention. Where other ports of entry are involved such as by ingestion or absorption through the skin, injection or topical methods, respectively, may be preferable. For topical application to the skin, carriers and formulations such as creams, ointments, lotions, and petrolatum products may be applied one or more times a day. Other methods of delivery, e.g., liposomal delivery or diffusion from a device impregnated with the composition, are known in the art. The compositions may be administered in a single bolus, multiple injections, or by continuous infusion (e.g., intravenously).

For the treatment of a cancerous tumor, compositions used in methods of the invention are generally formulated into a pharmaceutical composition that is administered by direct injection into the tumor to be treated, or administered into the tumor bed subsequent to tumor resection.

The compositions of the invention may be useful in preventing an infection by B. anthracis in individuals who have not yet been exposed to the bacterium. An example of such a prophylactic treatment involves administration of purified nucleic acids that hybridize to a nucleic acid encoding ATR and/or TEM8 polypeptide (e.g., antisense oligonucleotides) formulated in a pharmaceutical composition to an individual by any of the methods described above (e.g., oral, nasal administration). In such an individual, expression of ATR in the individual's cells is inhibited, and upon exposure to B. anthracis, binding of anthrax PA to the cells will be blocked, therefore preventing cellular infection and cellular death. Similarly, individuals who have been vaccinated for anthrax may also benefit from compositions of the invention. Inhibition of ATR expression by antisense nucleic acids (e.g., SEQ ID NOS: 1-17) may augment the anti-B. anthracis effects of the vaccine. Perhaps a most effective treatment for anthrax infection is administering to an infected or exposed individual antisense nucleic acids of the invention in combination with an antibiotic (e.g., ciprofloxacin).

Effective Doses

The compositions described above are preferably administered to a mammal (e.g., human) in an effective amount, that is, an amount capable of producing a desirable result in a treated subject (e.g., inhibiting expression of ATR and/or TEM8 in cells of the subject). Such a therapeutically effective amount can be determined as described below.

Toxicity and therapeutic efficacy of the compositions utilized in methods of the invention can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one animal depends on many factors, including the subject's size, body surface area, age, the particular composition to be administered, time and route of administration, general health, and other drugs being administered concurrently. Dosages of the disclosed antisense oligonucleotide compositions are to be efficacious and nontoxic, selected from a range of 1 ng/kg to 500 mg/kg and preferably less than 10 mg/kg. The selected dose is administered to a human when indicated anywhere from 1-6 or more times daily. The selected dose may also be administered to a human in a single dose. Intravenous or intraarterial administration generally requires lower doses since the drug is placed directly into the systemic circulation. It is expected that an appropriate dosage for intravenous administration of the compositions, if delivered via a rAAV vector, would be in the range of about 5μl/kg at 10¹³ rAAV particles and 50 μl/kg at 10¹² rAAV particles. As an example, for a 70 kg human a 3 ml injection of 10¹² particles is presently believed to be an appropriate dose. Dosages for nasal sprays typically range from about 10 mg to about 50 (total) or about 0.1 mg/kg to about 10 mg/kg. The dose therefore depends on the actual route of administration.

### EXAMPLES

The present invention is further illustrated by the following specific examples. The examples are provided for illustration only and are not intended to be construed as limiting the scope or content of the invention in any way.

### Example 1

### Selection of Antisense Sequences

To identify antisense sequences that could be used to disrupt PA binding to ATR, the GenBank database was searched for the mRNA sequence of Atr. Atr sequence was found in GenBank as Accession number AF421380. When choosing target sequence within Atr to which antisense oligonucleotides would hybridize, sequence encoding the VWA domain was avoided to prevent interference with VWA synthesis, as VWA deficiency is associated with bleeding in the host.

Antisense oligonucleotide lengths of 14-15-mers were selected initially because previous work indicated that the antisense oligonucleotides most frequently shown to be effective were 14-20 bases long. However, antisense sequences longer than 14-20 bases long (e.g., full-length cDNA) may also be useful because they can be inserted into plasmid or viral vectors.

When designing antisense molecules, two factors were considered. These factors are the affinity of a oligonucleotide for its target sequence, which is dependent on the number and composition of complementary bases, as well as the accessibility of the target sequence, which is dependent on the folding of the mRNA molecule. Antisense oligonucleotides with complicated secondary structure and self-dimerization potential are not preferred in applications of the invention. Self-dimerization and complicated secondary structures such as loops and hairpins in the antisense sequence prevent degradation, but also make hybridization with target mRNA more difficult. To examine the presence of the secondary structure, a computer program for designing PCR primers was employed. Characteristics of ideal antisense molecules are shown in Table 1.

TABLE 1
CHARACTERISTICS OF DEAL
ANTISENSE OLIGONUCLEOTDES (ODNS)

| | |
|---|---|
| 1. | The DNA sequence is specific and unique |
| 2. | Uptake into cells is efficient |
| 3. | The effect in cells is stable (for long-term treatment) or transient |
| | (for short-term treatment) |
| 4. | There is no non-specific binding to protein |
| 5. | Hybridization of the ODN is specific for the target DNA |
| 6. | The targeted protein and/or mRNA level is reduced |
| 7. | The ODN is not toxic |
| 8. | No inflammatory or immune response is induced |
| 9. | The ODN is more effective than appropriate sense and |
| | mismatch ODN controls |

Once antisense sequences with the appropriate characteristics were identified, selected antisense sequences were analyzed for uniqueness using a Blast Search. Matches were found between sequences 1-19 that targeted ATR and also the TEM 8 sequence (Carson-Walter, et al., Cancer Res. 61:6649-6655, 2001). A partial match with the human hydroxyacyl-Coenzyme A dehydrogenase type II (Yan, et al., Nature. 389:689-695, 1997) was also identified. It was therefore reasoned that the antisense sequences not only inhibit and/or interfere with the action of human ATR, but also inhibit TEM8, and possibly human hydroxyacyl-Coenzyme A dehydrogenase type II. Antisense sequences 1-17 (SEQ ID NOS: 1-17) were designed to hybridize to ATR and TEM8 based on this analysis. Antisense sequences 18 and 19 (SEQ ID NOS: 18, 19) were designed to hybridize particularly to human hydroxyacyl-Coenzyme A dehydrogenase type II.

Preferred regions of the mRNA for designing oligonucleotides which will hybridize to the mRNA were those which encompass or are near the AUG translation initiation codon, as well as those sequences which were substantially complementary to 5′ regions of the mRNA. Secondary structure analyses and target site selection considerations were performed using v. 4 of the OLIGO primer analysis software (Rychlik, 1997) and the BLASTN 2.0.5 algorithm software (Altschul, et al., 1997).

The sequences of SEQ ID NOS: 1-17 are preferred sequences for inhibiting anthrax toxin binding to host human cell receptors. The antisense compounds of the invention differ from native DNA by the modification of the phosphodiester backbone to extend the life of the antisense oligonucleotide in which the phosphate substitutents are replaced by phosphorothioates. One or both ends of the oligonucleotide may be substituted by one or more acridine derivatives which intercalates within DNA.

Selection of antisense sequences for inhibiting or mitigating infection of cells by anthrax was also based on an analysis of the Anthrax plasmid gene atxA as a target sequence. This gene expresses a transactivator of anthrax toxin synthesis. The analysis involved determination of secondary structure, melting temperature, binding energy, relative stability and relative inability to form dimers, hairpins or other secondary structures that reduced or prohibited specific binding to the target mRNA.

Using atxA sequence available in GenBank under accession number L13841, antisense sequences 20, 21 and 22 (SEQ ID NOS:20-22) were designed to hybridize to atxA mRNA. Antisense sequences 1-22 (SEQ ID NOS:20-22) were also designed to target the extracellular part of the protein, including the AUG translation initiation codon. While this part of the protein was initially examined for designing antisense sequences, antisense oligonucleotides to target other portions of the mRNA that promote synthesis of other parts of the protein may also be useful. There are many sites within the sequence that may be targeted. The most frequently targeted sites include the AUG translation initiation codon, but other sequences, including untranslated regions of the sequence, may also be useful.

### Example 2

### Testing Effectiveness in vitro

Functional assay for the anti-anthrax antisense treatment effect in vitro-macrophage lysis assay: Antrax toxin sensitive J774A. 1 macrophages are incubated with 0.02 μg/ml of LF (EC50 for lethal factor according to Gupta, et al, Infect Immun. 66:862-865, 1998, along with PA (1 μg/ml). The addition of LF causes lysis of macrophages. Antisense oligonucleotide (e.g., designed to hybridize to Atr murine homolog) is added at different time points and different concentrations. Three hours after adding LF and PA, viability is determined by adding 2-(2-methoxy-4-nitrophenyl)-3-(4-nitrophenyl)-5-(2,4-disulfophenyl)-2H-tetrazolium, monosodium salt (WST8) dye. After 1-4 hours in the incubator, absorbance at 450 nm (A450) is measured with the reference wavelength at 650 nm. The value of A450 is proportional to the amount of living cells. The A450 of LF, PA, and antisense-treated cells, therefore, should be higher than in the cells of macrophages treated only with LF and PA, in which more cells will die. Alternatively, human macrophages isolated using bronchoalveolar lavage could be used to test antisense oligonucleotides targeted to human sequence.

Functional Assay for the Effect of Anti-TEM8 (Anti-Tumor) Treatment-Cell Viability:

A tumor cell line such as human cervical cancer cells (HeLa), is pre-incubated with different concentrations of antisense oligonucleotide for 2-48 hr. Subsequently, 2-(2-methoxy-4-nitrophenyl)-3-(4-nitrophenyl)-5-(2,4-disulfophenyl)-2H-tetrazolium, monosodium salt (WST8) dye is added. After 1-4 hr in the incubator, an absorbance at 450 nm (A450) is measured with the reference wavelength at 650 nm. The A450 value is proportional to the amount of living cells, therefore in the groups with antisense-inhibited tumor cells, proliferation (the A450) should be lower then in the vehicle-treated tumor cells.

Receptor Binding Assay:

Target cells such as HeLa or macrophages are treated for different amounts of time with different concentrations of antisense oligonucleotide. Then, PA radiolabelled with iodide¹²⁵ is incubated with the target cells for 20 min at 4° C. Cells are washed to remove unbound PA and lysed with 100 mM NaOH. Radioactivity is measured using a γ-counter. The amount of the radioactivity is proportional to the amount of the receptors, and expressed as a percent of the control. Groups with the antisense-inhibited synthesis of the receptor will have less radioactivity than control cells.

### Example 3

### Testing Effectiveness In Vivo

Mouse Model:

In susceptible A/J mice, lethal infection by the spores of nonecapsulated, toxigenic Sterne strain of B. anthracis produces a disease similar to that caused by toxigenic and encapsulated B. anthracis. At the inoculation site, the mice develop an edematous exudate with large concentrations of bacilli and toxin, accompanied by systemic invasion and serum anthrax toxin levels increase in parallel with systemic bacterial concentrations and with the mortality rate. The mechanism has been associated with the deficiency of the complement component 5 (Welkos, et al., Microb Pathog. 1:53-69, 1988).

The susceptible mice may be used for testing the safety and efficacy of antisense treatment. Mice inoculated with a lethal dose of spores of nonecapsulated, toxigenic Sterne strain of B. anthracis are injected at different time points with different doses of antisense molecules (e.g., murine homolog of human sequence) and survival rates are measured.

Monkey Model: Monkeys may be used to test antisense molecules targeted to human nucleic acids. A method to infect rhesus macaques has been described (Fellows, et al., Vaccine 19:3241-3247, 2001). Subsequent to infection, monkeys are treated with different doses of antisense at different time points using different routes of delivery—intra-venous, bronchoalveolar, as well as nasal delivery. The blood of animals is drawn and tested for bacteremia. Survival rates are observed.

### Example 4

### Delivery of Antisense Oligonucleotides

Antisense may be delivered to a host (e.g., human) using a variety of delivery routes, including intra-venous, cutaneous, bronchoalveolar, and nasal delivery.

Intra-Venous Injection: A bolus or continuous injection is adminstered using. standard methods used in the clinics and hospitals.

Cutaneous Delivery: Cutaneous delivery is administered in the form of a cream, a lotion or an ointment, and applied one or more times a day.

Nasal Route: Antisense oligonucleotides are prepared in the form of an aerosol spray, and applied one or more times a day.

Bronchoalveolar Instillation: Bronchoscopy is performed as previously described (Koren, et al., Am. Rev. Respir. Dis. 139: 407-415, 1989). Before bronchoscopy, all subjects are premedicated intravenously with 0.6 mg atropine. The posterior pharynx is anesthetized by gargling with a saline solution containing 4% lidocaine, and the nasal passage is anesthetized with a lubricating jelly containing 2% lidocaine. The larynx, trachea, and bronchi are anesthetized with topical 2% lidocaine instilled through a fiberoptic bronchoscope (Olympus BF, type 1T20D; Olympus, Lake Success, N.Y. ) to control coughing.

To instill the antisense oligonucleotides into the distal airways and alveoli, the bronchoscope is passed to an identified subsegmental bronchus of the lingula but is not wedged. A sterile Teflon catheter is passed through the biopsy channel and then extended 4 to 5 cm beyond the tip of the bronchoscope into a subsegment of the lingula. Subjects are instructed to take deep, slow, regular breaths. A total of 10 ml sterile saline containing antisense molecules and liposomes is slowly instilled through the catheter coincident with inspirations to maximize aspiration of antisense into the alveolar region. This is followed by an additional 10 ml from a different syringe (for a total of 20 ml) with the intent of washing part remaining in airways into the alveoli. A total of 20 ml of sterile saline (without antisense) is instilled, as described, into the medial segment of the right middle lung lobe to serve as a control.

### Example 5

### Decreasing Expression of ATR/TEM8 using Antisense

The antisense oligonucleotide 5′-gccatggcccgcagc-3′(SEQ ID NO:7) directed to ATR and/or TEM8 was phosphorothioated and tested for its ability to decrease expression of ATR and/or TEM8.

Methods:

Tested cells—human lung fibroblasts CCD-Lu39 were transfected with different concentrations of the antisense oligonucleotide using Oligofectamine reagent. 24 or 48 h later cells were harvested using Trizol reagent and total RNA was prepared. Total RNA was digested with the DNase I, RNase -free, and reverse transcribed using random hexamers as primers. The cDNA was subjected to the real-time quantitative PCR using primers specific for ATR-TEM8 or 18S rRNA sequence. For quantitation, the amount of the ATR-TEM8 mRNA was normalized by the amount of 18S rRNA and expressed as a percent of the vehicle—Oligofectamine only sample. A statistical analysis was done using One-Way ANOVA and Tukey HSD Test.

Result of the 24 Hour Experiment:

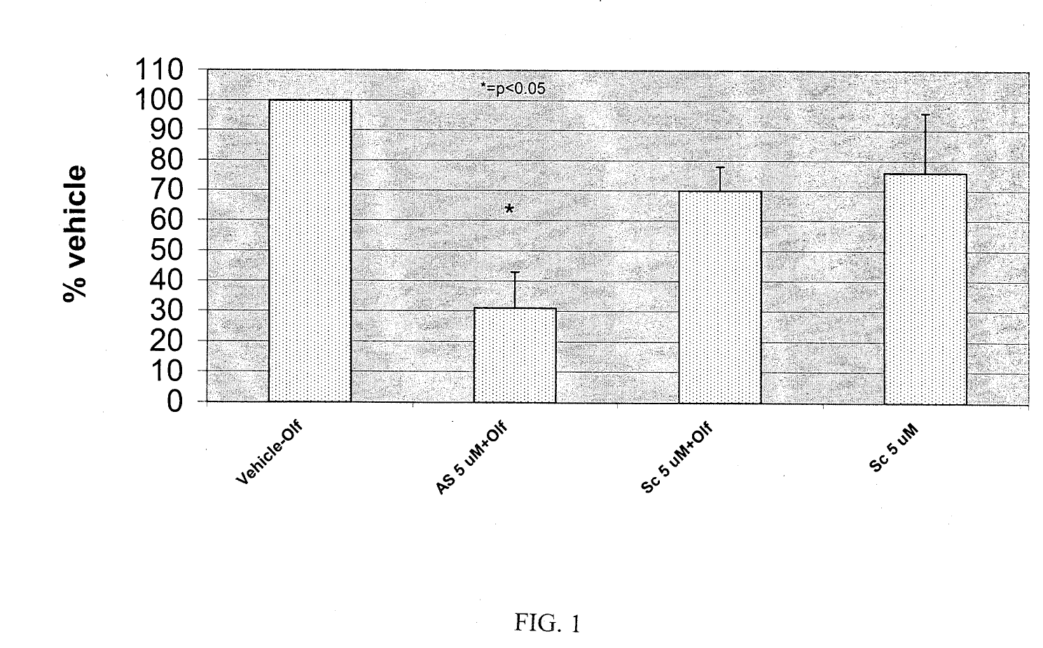

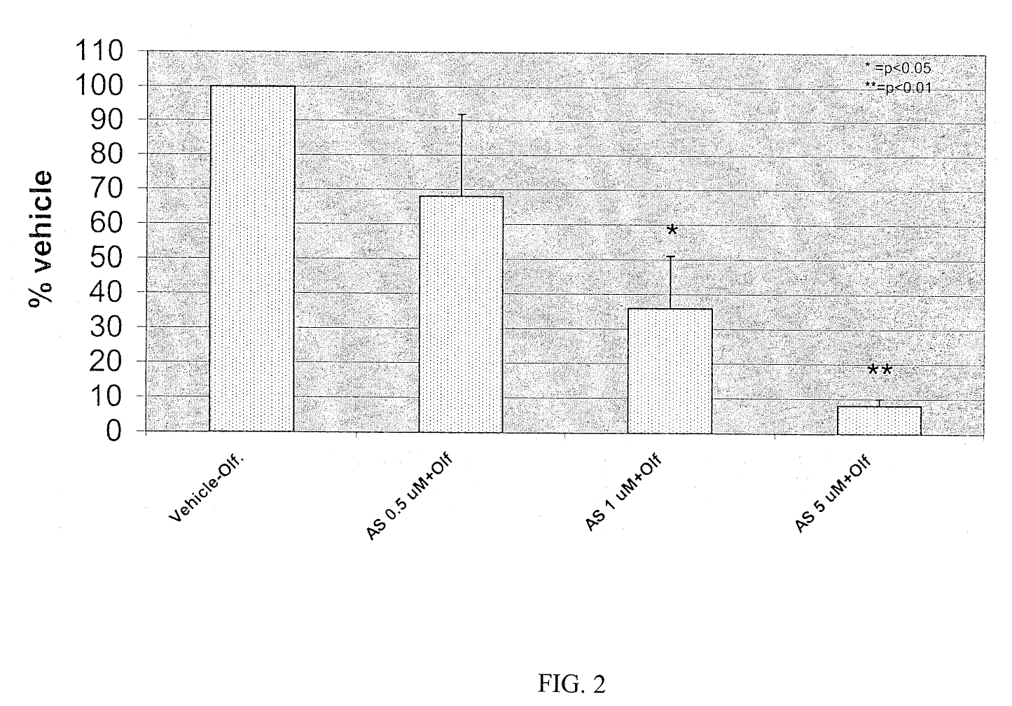

In the antisense-transfected human lung fibroblasts CCD-Lu39 cells, mRNA for the ATR-TEM8 was decreased by 69% (to 31% ±14, n=4, p<0.05) for 5 μM AS after 24 hours, as compared to the vehicle-treated control cells (FIG. 1). At the same time, the scrambled control alone or with the Oligofectamine did not changed significantly the ATR-TEM8 mRNA level (FIG. 2).

Result of the 48 Hour Experiment:

In the antisense-transfected CCD-Lu39 cells, mRNA for the ATR-TEM8 was decreased by 64% (to 36±15, n=3, p<0.05) for 1 μM AS, and by 92% (to 8%±2, n=4, p<0.01) for 5 μM AS after 48 hours, as compared to the vehicle-treated control cells (FIG. 2).

### Example 6

### Testing Antisense Oligonucleotides as Anti-tumor Treatment in Cell Viability Assay

Antisense oligonucleotide SEQ ID NO:7 was tested for its ability to decrease tumor cell viability in vitro. Methods: Tumor cells, like human cervical cancer cells HeLa and human lung adenocarcinoma A549, were transfected with different concentrations of the antisense oligonucleotide using Lipofectamine. After 24-96 hr, 2-(2-methoxy-4-nitrophenyl)-3-(4-nitrophenyl)-5-(2,4-disulfophenyl)-2H-tetrazolium, monosodium salt (WST8) dye was added to the cells to measure viability. After incubation of the cells with the WST8, an absorbance at 450 nm (A450) was measured with the reference wavelength at 650 nm. A value of A450/650 is proportional to the amount of living cells. The A450/650 in the groups with the antisense-transfected tumor cells was compared to the A450 in the vehicle (Lipofectamine)-treated tumor cells. Statistical analysis was done using One-Way ANOVA and Tukey HSD Test.

Results:

Viability of the cervix tumor cells HeLa was decreased to 56% (±7, n=4, p<0.01) 48 hours after the 10 μM AS transfection, as compared to the vehicle-treated tumor cells. Lung adenocarcinoma A549 cell viability was decreased to 49% (±10, n=4, p<0.01) 48 hr after the 1 μM AS transfection, as compared to the vehicle-treated tumor cells.

Other Embodiments

It is to be understood that while the invention has been described in conjunction with the detailed description thereof, the foregoing description is intended to illustrate and not limit the scope of the invention. For example, an additional embodiment of the invention relates to the inhibition of atxA expression using antisense nucleic acids (e.g., SEQ ID NOS: 20-22) that hybridize to mRNA encoding AtxA protein. This gene of the pXO1 plasmid encodes a transactivator of anthrax toxin synthesis. The AtxA protein appears to be crucial for bacterial virulence, toxin expression, capsule synthesis and escape of the bacteria from host macrophages (Uchida, et al., J. Bacteriol. 175: 5329-5338, 1993; Dai, et al., Mol Microbiol. 16: 1171-1181, 1995; Guignot, et al., FEMS Microbiol Lett. 147: 203-207, 1997; Dixon, et al., Cell Microbiol. 2: 453-463, 2000). Since the atxA gene is involved in so many aspects of the bacterial life cycle, its disruption even after infection will be beneficial.

What is claimed is:

1. A composition for inhibiting ATR/TEM8 expression in a cell, the composition comprising a purified antisense nucleic acid that hybridizes under stringent hybridization conditions to a polynucleotide that encodes a polypeptide selected from ATR and TEM8.

2. The composition of claim 1, wherein the antisense nucleic acid is selected from the group consisting of: SEQ ID NOS: 1-17.

3. The composition of claim 2, wherein the antisense nucleic acid is SEQ ID No:7.

4. The composition of claim 1, wherein the cell is a human cell.

5. The composition of claim 1, wherein the cell is a tumor cell.

6. The composition of claim 1, wherein the polypeptide is ATR.

7. The composition of claim 1, wherein the polypeptide is TEM8.

8. A vector comprising a nucleic acid sequence that encodes an antisense nucleic acid that hybridizes under stringent hybridization conditions to a polynucleotide that encodes a polypeptide selected from ATR and TEM8.

9. A method of modulating ATR or TEM8 expression in a cell, the method comprising the steps of:
(A) providing a cell expressing a molecule selected from ATR and TEM8; and
(B) contacting the cell with an agent that modulates expression of the molecule in the cell.

10. The method of claim 9, wherein the agent causes expression in the cell of an antisense nucleic acid that hybridizes under stringent hybridization conditions to a polynucleotide that encodes the molecule.

11. The method of claim 10, wherein the agent comprises the antisense nucleic acid.

12. The method of claim 9, wherein the molecule is ATR.

13. The method of claim 9, wherein the molecule is TEM8.

14. The method of claim 10, wherein the antisense nucleic acid is selected from the group consisting of: SEQ ID NOS: 1-17.

15. The method of claim 14, wherein the antisense nucleic acid is SEQ ID NO:7.

16. The method of claim 9, wherein the cell is a human cell.

17. The method of claim 9, wherein the cell is a tumor cell.

18. A method of modulating tumor cell viability, the method comprising the steps 5 of:
(A) providing a tumor cell expressing TEM8; and
(B) administering to the tumor cell a composition comprising an agent that modulates expression of TEM8 in the cell.

19. The method of claim 18, wherein the agent causes expression in the cell of an antisense nucleic acid that hybridizes under stringent hybridization conditions to a polynucleotide that encodes TEM8.

20. The method of claim 19, wherein the agent comprises the antisense nucleic acid.

21. The method of claim 19, wherein the antisense nucleic acid is selected from the group consisting of: SEQ ID NOS: 1-17.

22. The method of claim 21, wherein the antisense nucleic acid is SEQ ID NO:7.

23. The method of claim 18, wherein the cell is a human cell.


SEQUENCE LISTING (22 sequences)

SEQ ID NO: 1 - DNA, 15 bp, Homo sapiens
ttcctcgcgggtcct

SEQ ID NO: 2 - DNA, 15 bp, Homo sapiens
cagggacgcgccatc

SEQ ID NO: 3 - DNA, 15 bp, Homo sapiens
cgccacgaccctcag

SEQ ID NO: 4 - DNA, 14 bp, Homo sapiens
gctccgcgaactcg

SEQ ID NO: 5 - DNA, 15 bp, Homo sapiens
tccgctccttcccac

SEQ ID NO: 6 - DNA, 14 bp, Homo sapiens
gggagagcagggtc

SEQ ID NO: 7 - DNA, 15 bp, Homo sapiens
gccatggcccgcagc

SEQ ID NO: 8 - DNA, 15 bp, Homo sapiens
ccgccgtggccatgg

SEQ ID NO: 9 - DNA, 15 bp, Homo sapiens
ctccgccgtggccat

SEQ ID NO: 10 - DNA, 15 bp, Homo sapiens
agggctctccgctcc

SEQ ID NO: 11 - DNA, 15 bp, Homo sapiens
tggaagccgatgccg

SEQ ID NO: 12 - DNA, 15 bp, Homo sapiens
gccaaagagagccac

SEQ ID NO: 13 - DNA, 15 bp, Homo sapiens
gatgagcaccagagt

SEQ ID NO: 14 - DNA, 14 bp, Homo sapiens
cccttgcccggcgc

SEQ ID NO: 15 - DNA, 15 bp, Homo sapiens
atcctccctgcgtcc

SEQ ID NO: 16 - DNA, 15 bp, Homo sapiens
ccgtagcaggctgga

SEQ ID NO: 17 - DNA, 15 bp, Homo sapiens
aaatccgccgtagca

SEQ ID NO: 18 - DNA, 15 bp, Homo sapiens
acacgctgctgccat

SEQ ID NO: 19 - DNA, 15 bp, Homo sapiens
tgctgccatcttgtc

SEQ ID NO: 20 - DNA, 15 bp, Bacillus anthracis
catgtctataattga

SEQ ID NO: 21 - DNA, 15 bp, Bacillus anthracis
tatcggtgttagcat

SEQ ID NO: 22 - DNA, 15 bp, Bacillus anthracis
ctgcgacctgtagat